(19) United States
(12) Patent Application Publication
(10) Pub. No.: US 2025/0025694 A1
(43) Pub. Date: Jan. 23, 2025

(54) SYSTEM FOR ELECTRICAL ACTIVITY SENSING

(71) Applicant: SCENERAY CO., LTD., Suzhou (CN)

(72) Inventors: Bin Liu, Suzhou (CN); Weiran Zhu, Suzhou (CN); Jinghua Chen, Suzhou (CN)

(73) Assignee: SCENERAY CO., LTD., Suzhou, JS (CN)

(21) Appl. No.: 18/689,991

(22) PCT Filed: Sep. 14, 2021
(86) PCT No.: PCT/CN2021/118191

§ 371 Date: Mar. 7, 2024

(30) Foreign Application Priority Data
Sep. 9, 2021 (CN) 202111054819.2

(51) Int. Cl. A61N 1/36

(52) U.S. Cl. CPC A61N 1/3606; A61N 1/3614

(57) ABSTRACT

Provided is a system (40) including multiple electrodes, a treatment delivery circuit (10), a sensing circuit (20) and a controller (30). The multiple electrodes are capable of being positioned within a brain of a patient to deliver a treatment to the patient or sense an electrical activity. The treatment delivery circuit (10) is operably coupled to the multiple electrodes to deliver the treatment to the patient. The sensing circuit (20) is operably coupled to the multiple electrodes to sense the electrical activity. The controller (30) includes a processing circuit system operably coupled to the treatment delivery circuit (10) and the sensing circuit (20). The controller (30) is configured to: control, through the treatment delivery circuit (10), one or more electrodes among the multiple electrodes to deliver the treatment to the patient; sense, through the sensing circuit (20), potentials of the multiple electrodes in a process of delivering the treatment; and calculate, based on the potentials of the multiple electrodes, a difference value between potentials of any two electrodes among the multiple electrodes to obtain a voltage between the any two electrodes. The system (40) can collect the electrical signal of the deep brain of the patient in a case where the patient receives the stimulus.

### TECHNICAL FIELD

The present application relates to the technical field of implantable medical systems, and in particular, to a system for an electrical activity sensing, especially, a system for performing an electrical activity sensing while delivering a treatment.

### BACKGROUND

An implantable medical system typically includes an implantable electrical nerve stimulation system, an implantable electrical cardiac stimulation system, and an implantable drug infusion system.

The implantable electrical nerve stimulation system is used as an example, this system mainly includes a pulse generator implanted into a body, an electrode, and a control device outside the body. The pulse generator is connected to the electrode so as to transmit a pulse generated by the pulse generator to the electrode, and a pulse signal generated by the pulse generator is transmitted to a specific nerve part by the electrode to perform the electric stimulation, so that the function of the human body is restored to the normal operation state.

Currently, an electrical signal of deep brain of a patient is collected in a case where the patient does not receive a stimulus, whereby the collected electrical signal cannot reflect a brain condition of the patient during the stimulus.

### SUMMARY

According to an embodiment disclosed in the present application, a system for an electrical activity sensing is provided. The system includes multiple electrodes, a treatment delivery circuit, a sensing circuit and a controller. The multiple electrodes are capable of being positioned within a brain of a patient to deliver a treatment to the patient or sense an electrical activity. The treatment delivery circuit is operably coupled to the multiple electrodes to deliver the treatment to the patient. The sensing circuit is operably coupled to the multiple electrodes to sense the electrical activity. The controller includes a processing circuit system operably coupled to the treatment delivery circuit and the sensing circuit. The controller is configured to: control, through the treatment delivery circuit, one or more electrodes among the multiple electrodes to deliver the treatment to the patient; sense, through the sensing circuit, potentials of the multiple electrodes in a process of delivering the treatment; and calculate, based on the potentials of the multiple electrodes, a difference value between potentials of any two electrodes among the multiple electrodes to obtain a voltage between the any two electrodes.

In an embodiment, the controller is further configured to: control, through the treatment delivery circuit, one electrode of the multiple electrodes to deliver the treatment to the patient; and acquire, based on voltages between the one electrode and other electrodes of the multiple electrodes, a voltage decay curve in which the one electrode delivers the treatment to the patient, and one or more of impedances of brain tissue between the one electrode and the other electrodes of the multiple electrodes.

In an embodiment, that the voltage decay curve in which the one electrode delivers the treatment to the patient is acquired based on the voltages between the one electrode and the other electrodes of the multiple electrodes includes: the voltage decay curve in which the one electrode delivers the treatment to the patient is plotted by using distances between the one electrode and the other electrodes of the multiple electrodes as a horizontal axis and using voltages between the one electrode and the other electrodes of the multiple electrodes as a vertical axis.

In an embodiment, where the controller is further configured to: determine a first electrode combination including the one electrode based on the voltage decay curve in which the one electrode delivers the treatment to the patient, and the one or more of the impedances of the brain tissue between the one electrode and the other electrodes of the multiple electrodes; and control, through the treatment delivery circuit, the first electrode combination to deliver the treatment to the patient.

In an embodiment, the controller is further configured to: determine a second electrode combination with a slowest decay based on a voltage decay curve in which each electrode of the multiple electrodes delivers the treatment to the patient; and control, through the treatment delivery circuit, the second electrode combination to deliver the treatment to the patient.

In an embodiment, the electrical activity that is capable of being sensed by the multiple electrodes includes an electrical activity delivering the treatment to the patient and a bioelectrical activity of the patient.

In an embodiment, the system further includes a filter, and the filter is operably coupled between the sensing circuit and the controller to filter out the bioelectrical activity of the patient sensed by the multiple electrodes or the electrical activity delivering the treatment to the patient sensed by the multiple electrodes.

In an embodiment, the system further includes a signal amplification circuit, and the signal amplification circuit is operably coupled between the sensing circuit and the controller to amplify the bioelectrical activity of the patient sensed by the multiple electrodes.

In an embodiment, the system further includes a signal reduction circuit, and the signal reduction circuit is operably coupled between the sensing circuit and the controller to reduce the electrical activity delivering the treatment to the patient sensed by the multiple electrodes.

In an embodiment, the bioelectrical activity of the patient is a single cell electrical activity, a nuclei electrical activity, or a nuclei local electrical activity.

According to an embodiment disclosed in the present application, a system is provided. The system includes: a device configured to receive potentials of multiple electrodes in response to determining that one or more electrodes of the multiple electrodes delivers a treatment to a patient; and a device configured to calculate a difference value between potentials of any two electrodes among the multiple electrodes based on the potentials of the multiple electrodes to obtain a voltage between the any two electrodes. The multiple electrodes are capable of being positioned in a brain of the patient to deliver the treatment to the patient or sense an electrical activity.

In an embodiment, the system further includes: a device configured to control one electrode of the multiple electrodes to deliver the treatment to the patient; and a device configured to calculate, based on voltages between the one electrode and other electrodes of the multiple electrodes, a voltage decay curve in which the one electrode delivers the treatment to the patient, and one or more of impedances of brain tissue between the one electrode and the other electrodes of the multiple electrodes.

In an embodiment, the device configured to calculate, based on the voltages between the one electrode and the other electrodes of the multiple electrodes, the voltage decay curve in which the one electrode delivers the treatment to the patient, and the one or more of the impedances of the brain tissue between the one electrode and the other electrodes of the multiple electrodes are further configured to: plot the voltage decay curve in which the one electrode delivers the treatment to the patient by using distances between the one electrode and the other electrodes of the multiple electrodes as a horizontal axis and using voltages between the one electrode and the other electrodes of the multiple electrodes as a vertical axis.

In an embodiment, the system further includes: a device configured to determine a first electrode combination including the one electrode based on the voltage decay curve in which the one electrode delivers the treatment to the patient, and the one or more of the impedances of the brain tissue between the one electrode and the other electrodes of the multiple electrodes; and a device configured to control the first electrode combination to deliver the treatment to the patient.

In an embodiment, the system further includes: a device configured to determine a second electrode combination with a slowest decay based on a voltage decay curve in which each electrode of the multiple electrodes delivers the treatment to the patient; and a device configured to control the second electrode combination to deliver the treatment to the patient.

### BRIEF DESCRIPTION OF DRAWINGS

Further features, properties and various advantages of the subject matter disclosed in the present application will become more apparent from the following specific implementation modes and the accompanying drawings, in which,

FIG. 1 is a schematic diagram of a simplified block diagram of a system according to an embodiment of the present application.

### DETAILED DESCRIPTION

The present application is further described in conjunction with the accompanying drawings and the specific implementation modes. It should be noted that, in the case of no conflict, the following described embodiments or technical features may be randomly combined to form a new embodiment.

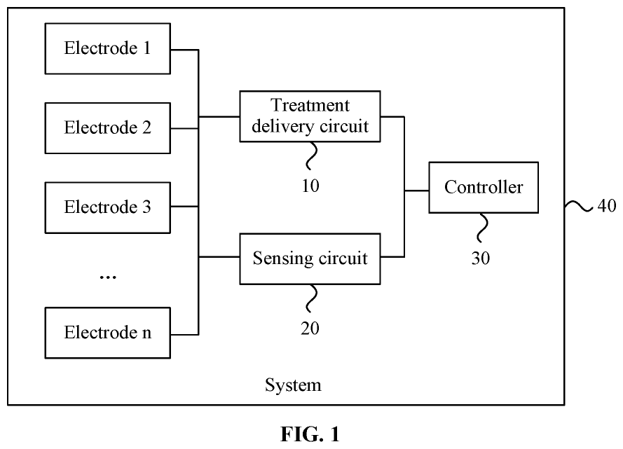

Referring to FIG. 1, an embodiment of the present application provides a system 40. The system includes multiple electrodes, a treatment delivery circuit 10, a sensing circuit 20 and a controller 30. The multiple electrodes are capable of being positioned within a brain of a patient to deliver a treatment to the patient or sense an electrical activity. The treatment delivery circuit 10 is operably coupled to the multiple electrodes to deliver the treatment to the patient. The sensing circuit 20 is operably coupled to the multiple electrodes to sense the electrical activity. The controller 30 includes a processing circuit system operably coupled to the treatment delivery circuit 10 and the sensing circuit 20. The controller 30 is configured to: control, through the treatment delivery circuit 10, one or more electrodes among the multiple electrodes to deliver the treatment to the patient; sense, through the sensing circuit 20, potentials of the multiple electrode in a process of delivering the treatment; and calculate, based on the potentials of the multiple electrodes, a difference value between potentials of any two electrodes among the multiple electrodes to obtain a voltage between the any two electrodes.

The treatment delivery circuit 10 may include a pulse generator and an extension wire connected to the pulse generator, one end of the extension wire is connected to the pulse generator, and another end of the extension wire is connected to the electrode, so as to transmit the pulse generated by the pulse generator to the electrode, and deliver the treatment to the patient by using the electrode. The pulse generator may be coupled to the controller 30.

The controller 30 may include a memory and a processor. A size, a shape, and a position of the controller 30 are not limited in the present application. The control function of the controller 30 may be implemented by an MPU, an MCU, a DSP, an FPGA, or any combination thereof, and the controller 30 may support the wireless upgrading of the embedded program in an integrated chip manner.

According to the system 40 of the present application, when the electrical activity is sensed, one or more electrodes among the multiple electrodes are controlled through the treatment delivery circuit 10 to deliver the treatment to the patient. Moreover, the potentials of the multiple electrodes are sensed through the sensing circuit 20, and the difference value between the potentials of the any two electrodes among the multiple electrodes are calculated based on the potentials of the multiple electrodes to obtain the voltage between the any two electrodes.

Since the electrical activity sensing is performed in synchronization with the delivery treatment of the patient, the measured voltage may reflect, to some extent, a brain condition of the patient when the patient receives the delivery treatment, which has a greater reference value for the subsequent treatment of the patient.

In an embodiment, the controller 30 may be further configured to: control, through the treatment delivery circuit 10, one electrode of the multiple electrodes to deliver the treatment to the patient; and acquire, based on voltages between the one electrode and other electrodes of the multiple electrodes, a voltage decay curve in which the one electrode delivers the treatment to the patient, and one or more of impedances of brain tissue between the one electrode and the other electrodes of the multiple electrodes.

Therefore, the voltage decay curve in which the one electrode delivers the treatment to the patient may be acquired, so as to more intuitively reflect the brain condition of the patient when the patient receives the delivery treatment. The one or more of the impedances of the brain tissue between the one electrode and the other electrodes of the multiple electrodes may be acquired, so as to reflect a state of the brain tissue of the patient when the patient receives the delivery treatment to some extent.

In an embodiment, that the voltage decay curve in which the one electrode delivers the treatment to the patient is acquired based on the voltages between the one electrode and the other electrodes of the multiple electrodes includes: the voltage decay curve in which the one electrode delivers the treatment to the patient is plotted by using distances between the one electrode and the other electrodes of the multiple electrodes as a horizontal axis and using voltages between the one electrode and the other electrodes of the multiple electrodes as a vertical axis.

Therefore, the voltage decay curve in which the one electrode delivers the treatment to the patient is plotted by using the distances between the one electrode and the other electrodes of the multiple electrodes as the horizontal axis and using the voltages between the one electrode and the other electrodes of the multiple electrodes as the vertical axis, so that a brain condition of a treatment site in which the one electrode is located and a site corresponding to the other electrodes when the patient receives the delivery treatment may be reflected.

In an embodiment, the controller 30 may be further configured to: determine a first electrode combination including the one electrode based on the voltage decay curve in which the one electrode delivers the treatment to the patient, and the one or more of the impedances of the brain tissue between the one electrode and the other electrodes of the multiple electrodes; and control, through the treatment delivery circuit 10, the first electrode combination to deliver the treatment to the patient.

Therefore, after the voltage decay curve in which the one electrode delivers the treatment to the patient, and the one or more of the impedances of the brain tissue between the one electrode and the other electrodes of the multiple electrodes are obtained, the voltage decay curve and the impedances of the brain tissue may be used as reference, and the first electrode combination is controlled through the treatment delivery circuit 10 to deliver the treatment to the patient.

In an embodiment, the controller 30 may be further configured to: determine a second electrode combination with a slowest decay based on a voltage decay curve in which each electrode of the multiple electrodes delivers the treatment to the patient; and control, through the treatment delivery circuit, the second electrode combination to deliver the treatment to the patient.

Therefore, the second electrode combination with the slowest decay may be determined based on the voltage decay curve in which each electrode of the multiple electrodes delivers the treatment to the patient, and the second electrode combination is controlled through the treatment delivery circuit 10 to deliver the treatment to the patient. Generally, the slower the voltage of the electrode decays, the better the therapeutic effect.

In an embodiment, the electrical activity that is capable of being sensed by the multiple electrodes may include an electrical activity delivering the treatment to the patient and a bioelectrical activity of the patient.

Generally, the signal strength of the electrical activity that delivers the treatment to the patient is larger, and the bioelectrical activity signal of the patient is relatively weak, typically on the order of microvolts.

In an embodiment, the system 40 may further include a filter, and the filter is operably coupled between the sensing circuit 20 and the controller 30 to filter out the bioelectrical activity of the patient sensed by the multiple electrodes or the electrical activity delivering the treatment to the patient sensed by the multiple electrodes.

In one aspect, the filter is provided so that the bioelectrical activity of the patient may be filtered out, whereby the calculated voltage between the electrodes may reflect the strength of the treatment delivered to the patient. On the other hand, the electrical activity that delivers the treatment to the patient may be filtered by using the filter, whereby the calculated voltage between the electrodes may reflect the brain electrical signal strength of the patient.

In an embodiment, the system 40 may further include a signal amplification circuit, and the signal amplification circuit is operably coupled between the sensing circuit 20 and the controller 30 to amplify the bioelectrical activity of the patient sensed by the multiple electrodes. The magnification may be 10 times, 100 times, or 1000 times.

The signal amplification circuit is provided so that the bioelectrical activity of the patient sensed by the multiple electrodes may be amplified for ease of the subsequent calculation.

In an embodiment, the system 40 may further include a signal reduction circuit, and the signal reduction circuit is operably coupled between the sensing circuit 20 and the controller 30 to reduce the electrical activity delivering the treatment to the patient sensed by the multiple electrodes. The reduction multiple may be 2 times, 3 times, or 5 times.

The signal reduction circuit is provided so that the electrical activity delivering the treatment to the patient sensed by the multiple electrodes may be reduced. Generally, an apparatus for measuring the potential of the electrode is relatively precise, and the measurement range is relatively small. The electrical activity delivering the treatment to the patient sensed by the multiple electrodes are reduced, so that the measured potential values may be avoided from exceeding the measurement range.

In an embodiment, the bioelectrical activity of the patient is a single cell electrical activity, a nuclei electrical activity, or a nuclei local electrical activity.

Generally, an electrode required for collecting a single cell electrical signal is different from an electrode required for collecting a local field potential. A single cell requires a small electrode or microelectrode, and the signal collected by the single cell has a high amplitude and a high frequency. The local field potential needs a normal electrode or a macro electrode, the collected signal has a low amplitude and a low frequency, and the size of the normal electrode is, such as, 6 square millimeters.

The system of the present application may sense the single cell electrical activity, the nuclei electrical activity or the nuclei local electrical activity by using the multiple electrodes.

An embodiment of the present application further provides a system. The system includes: a device configured to receive potentials of multiple electrodes in response to determining that one or more electrodes of the multiple electrodes delivers a treatment to a patient; and a device configured to calculate a difference value between potentials of any two electrodes among the multiple electrodes based on the potentials of the multiple electrodes to obtain a voltage between the any two electrodes. The multiple electrodes are capable of being positioned in a brain of the patient to deliver the treatment to the patient or sense an electrical activity.

Thus, the electrical activity may be sensed while the delivery treatment is performed on the patient, and the measured voltage may reflect, to some extent, a brain condition of the patient when the patient receives the delivery treatment, which has a greater reference value for the subsequent treatment of the patient.

In an embodiment, the system may further include: a device configured to control one electrode of the multiple electrodes to deliver the treatment to the patient; and a device configured to calculate, based on voltages between the one electrode and other electrodes of the multiple electrodes, a voltage decay curve in which the one electrode delivers the treatment to the patient, and one or more of impedances of brain tissue between the one electrode and the other electrodes of the multiple electrodes.

Therefore, the voltage decay curve in which the one electrode delivers the treatment to the patient may be acquired, so as to more intuitively reflect the brain condition of the patient when the patient receives the delivery treatment. The impedances of the brain tissue between the one electrode and the other electrodes of the multiple electrodes may be acquired, so as to reflect a state of the brain tissue of the patient when the patient receives the delivery treatment to some extent.

In an embodiment, the device configured to calculate, based on the voltages between the one electrode and the other electrodes of the multiple electrodes, the voltage decay curve in which the one electrode delivers the treatment to the patient, and the one or more of the impedances of the brain tissue between the one electrode and the other electrodes of the multiple electrodes are further configured to: plot the voltage decay curve in which the one electrode delivers the treatment to the patient by using distances between the one electrode and the other electrodes of the multiple electrodes as a horizontal axis and using voltages between the one electrode and the other electrodes of the multiple electrodes as a vertical axis.

Therefore, the voltage decay curve in which the one electrode delivers the treatment to the patient is plotted by using the distances between the one electrode and the other electrodes of the multiple electrodes as the horizontal axis and using the voltages between the one electrode and the other electrodes of the multiple electrodes as the vertical axis, so that a brain condition of a treatment site in which the one electrode is located and a site corresponding to the other electrodes when the patient receives the delivery treatment may be reflected.

In an embodiment, the system may further include: a device configured to determine a first electrode combination including the one electrode based on the voltage decay curve in which the one electrode delivers the treatment to the patient, and the one or more of the impedances of the brain tissue between the one electrode and the other electrodes of the multiple electrodes; and a device configured to control the first electrode combination to deliver the treatment to the patient.

Therefore, after the voltage decay curve in which the one electrode delivers the treatment to the patient, and the one or more of the impedances of the brain tissue between the one electrode and the other electrodes of the multiple electrodes are obtained, the attenuation and the impedances of the brain tissue may be used as reference, and the first electrode combination is controlled to deliver the treatment to the patient

In an embodiment, the system may further include: a device configured to determine a second electrode combination with a slowest decay based on a voltage decay curve in which each electrode of the multiple electrodes delivers the treatment to the patient; and a device configured to control the second electrode combination to deliver the treatment to the patient.

Therefore, the second electrode combination with the slowest decay may be determined based on the voltage decay curve in which each electrode of the multiple electrodes delivers the treatment to the patient, and the second electrode combination is controlled to deliver the treatment to the patient. Generally, the slower the voltage of the electrode decays, the better the therapeutic effect.

The present application is described in view of the purpose of use, efficiency, progress, novelty and the like, and complies with the functional improvement and use requirements highlighted by the Patent Law, the above Specification and drawings of the present application are only preferred embodiments of the present application, and are not intended to limit the present application.

Therefore, any equivalent replacement or modification that is similar to or identical to the construction, device, feature and the like of the present application, that is, any equivalent replacement or modification that is made according to the scope of the present application shall fall within the scope of protection of the present application.

1. A system, comprising:
a plurality of electrodes, wherein the plurality of electrodes is capable of being positioned within a brain of a patient to deliver a treatment to the patient or sense an electrical activity;
a treatment delivery circuit, wherein the treatment delivery circuit is operably coupled to the plurality of electrodes to deliver the treatment to the patient;
a sensing circuit, wherein the sensing circuit is operably coupled to the plurality of electrodes to sense the electrical activity; and
a controller, wherein the controller comprises a processing circuit system operably coupled to the treatment delivery circuit and the sensing circuit, and the controller is configured to perform: controlling, through the treatment delivery circuit, one or more electrodes among the plurality of electrodes to deliver the treatment to the patient; sensing, through the sensing circuit, potentials of the plurality of electrodes in a process of delivering the treatment; and
calculating, based on the potentials of the plurality of electrodes, a difference value between potentials of two electrodes among the plurality of electrodes to obtain a voltage between the two electrodes.

2. The system of claim 1, wherein the controller is further configured to perform:
controlling, through the treatment delivery circuit, one electrode of the plurality of electrodes to deliver the treatment to the patient; and
acquiring, based on voltages between the one electrode and other electrodes of the plurality of electrodes, a voltage decay curve in which the one electrode delivers the treatment to the patient, and acquiring one or more of impedances of brain tissue between the one electrode and the other electrodes of the plurality of electrodes.

3. The system of claim 2, wherein acquiring, based on voltages between the one electrode and other electrodes of the plurality of electrodes, a voltage decay curve in which the one electrode delivers the treatment to the patient comprises: plotting the voltage decay curve in which the one electrode delivers the treatment to the patient by using distances between the one electrode and the other electrodes of the plurality of electrodes as a horizontal axis and using voltages between the one electrode and the other electrodes of the plurality of electrodes as a vertical axis.

4. The system of claim 2, wherein the controller is further configured to perform:
determining a first electrode combination comprising the one electrode based on the voltage decay curve in which the one electrode delivers the treatment to the patient, and the one or more of the impedances of the brain tissue between the one electrode and the other electrodes of the plurality of electrodes; and
controlling, through the treatment delivery circuit, the first electrode combination to deliver the treatment to the patient.

5. The system of claim 2, wherein the controller is further configured to perform:
determining a second electrode combination with a slowest decay based on a voltage decay curve in which each electrode of the plurality of electrodes delivers the treatment to the patient;
and control, through the treatment delivery circuit, the second electrode combination to deliver the treatment to the patient.

6. The system of claim 1, wherein the electrical activity that is capable of being sensed by the plurality of electrodes comprises an electrical activity delivering the treatment to the patient and a bioelectrical activity of the patient.

7. The system of claim 6, further comprising a filter, wherein the filter is operably coupled between the sensing circuit and the controller to filter out the bioelectrical activity of the patient sensed by the plurality of electrodes or the electrical activity delivering the treatment to the patient sensed by the plurality of electrodes.

8. The system of claim 6, further comprising a signal amplification circuit, wherein the signal amplification circuit is operably coupled between the sensing circuit and the controller to amplify the bioelectrical activity of the patient sensed by the plurality of electrodes.

9. The system of claim 6, further comprising a signal reduction circuit, wherein the signal reduction circuit is operably coupled between the sensing circuit and the controller to reduce the electrical activity delivering the treatment to the patient sensed by the plurality of electrodes.

10. The system of claim 6, wherein the bioelectrical activity of the patient is a single cell electrical activity, a nuclei electrical activity, or a nuclei local electrical activity.

11. A system, comprising:
a device configured to receive potentials of a plurality of electrodes in response to determining that one or more electrodes of the plurality of electrodes delivers a treatment to a patient; and
a device configured to calculate a difference value between potentials of two electrodes among the plurality of electrodes based on the potentials of the plurality of electrodes to obtain a voltage between the two electrodes;
wherein the plurality of electrodes is capable of being positioned in a brain of the patient to deliver the treatment to the patient or sense an electrical activity.

12. The system of claim 11, further comprising:
a device configured to control one electrode of the plurality of electrodes to deliver the treatment to the patient; and
a device configured to calculate, based on voltages between the one electrode and other electrodes of the plurality of electrodes, a voltage decay curve in which the one electrode delivers the treatment to the patient, and one or more of impedances of brain tissue between the one electrode and the other electrodes of the plurality of electrodes.

13. The system of claim 12, wherein the device configured to calculate, based on the voltages between the one electrode and the other electrodes of the plurality of electrodes, the voltage decay curve in which the one electrode delivers the treatment to the patient, and the one or more of the impedances of the brain tissue between the one electrode and the other electrodes of the plurality of electrodes is further configured to: plot the voltage decay curve in which the one electrode delivers the treatment to the patient by using distances between the one electrode and the other electrodes of the plurality of electrodes as a horizontal axis and using voltages between the one electrode and the other electrodes of the plurality of electrodes as a vertical axis.

14. The system of claim 12, further comprising:
a device configured to determine a first electrode combination comprising the one electrode based on the voltage decay curve in which the one electrode delivers the treatment to the patient, and the one or more of the impedances of the brain tissue between the one electrode and the other electrodes of the plurality of electrodes; and
a device configured to control the first electrode combination to deliver the treatment to the patient.

15. The system of claim 12, further comprising:
a device configured to determine a second electrode combination with a slowest decay based on a voltage decay curve in which each electrode of the plurality of electrodes delivers the treatment to the patient; and
a device configured to control the second electrode combination to deliver the treatment to the patient.

16. The system of claim 2, wherein the electrical activity that is capable of being sensed by the plurality of electrodes comprises an electrical activity delivering the treatment to the patient and a bioelectrical activity of the patient.

17. The system of claim 3, wherein the electrical activity that is capable of being sensed by the plurality of electrodes comprises an electrical activity delivering the treatment to the patient and a bioelectrical activity of the patient.

18. The system of claim 4, wherein the electrical activity that is capable of being sensed by the plurality of electrodes comprises an electrical activity delivering the treatment to the patient and a bioelectrical activity of the patient.

19. The system of claim 5, wherein the electrical activity that is capable of being sensed by the plurality of electrodes comprises an electrical activity delivering the treatment to the patient and a bioelectrical activity of the patient.

20. The system of claim 1, wherein the treatment delivery circuit comprises a pulse generator and extension wires connected to the pulse generator, one end of each of the extension wires is connected to the pulse generator, and another end of each of the extension wires is connected to a respective one of the plurality of electrodes to transmit pulses generated by the pulse generator to the respective one of the plurality of electrodes, and deliver the treatment to the patient by using the plurality of electrodes.
